(19) United States
(12) Patent Application Publication
(10) Pub. No.: US 2007/0082073 A1
(43) Pub. Date: Apr. 12, 2007

(54) Catechin Adjuvants

(75) Inventors: Alberto van Olphen, Tampa, FL (US); Herman Friedman, Tampa, FL (US); Neil Shaw, Tampa, FL (US); Nicholas Burdash, Tampa, FL (US); James Roger, Tampa, FL (US)

Correspondence Address: SMITH HOPEN, PA, 180 PINE AVENUE NORTH, OLDSMAR, FL, 34677, US

(73) Assignee: UNIVERSITY OF SOUTH FLORIDA, Tampa, FL (US)

(21) Appl. No.: 11/419,101

(22) Filed: May 18, 2006

Related U.S. Application Data
(60) Provisional application No. 60/594,916, filed on May 18, 2005.

(51) Int. Cl. A61K 36/82; A61K 31/7048; A61K 31/704; A61K 31/4745; A61K 31/66

(52) U.S. Cl. 424/729; 514/34; 514/109; 514/27; 514/283

(57) ABSTRACT

A combination cancer therapy based upon catechins, the major biologically active polyphenol in plant products, including green tea extract, and one or more chemotherapeutic agents. A common complication of cancer chemotherapy is neutropenia, and in spite of advances in its prophylactic management is a major cause of risk for the development of serious microbial infections leading to increased morbidity and mortality in both humans and animals. The use of cathechins such as those found in green tea (Camellia sinensis), including but not limited to epigallocatechin gallate (EGCG) as nontoxic adjuvant to aid in the prevention of opportunistic microbial infections in patients undergoing immunosuppressive chemotherapy is a novel application. Also contemplated are methods using catechins to ameliorate the immunosuppressive effects of cancer chemotherapy by administering the compound to a patient in need thereof.

### CROSS REFERENCE TO RELATED APPLICATIONS

This application claims priority to currently pending U.S. Provisional Patent Application 60/594,916, entitled, “Catechins as Nontoxic Adjuvants for Cancer Chemotherapy”, filed May 18, 2005, the contents of which are herein incorporated by reference.

### FIELD OF INVENTION

This invention relates to adjuvants. More particularly, this invention relates to catechins as nontoxic adjuvants for cancer chemotherapy.

### BACKGROUND OF THE INVENTION

There is a wide commercial need for inexpensive small molecular weight immunoenhancing drugs to enhance the immune response of individuals treated with antimetabolite anti-cancer drugs, which are immunosuppressive. We are using as the preclinical prototype pet canines treated for lymphoma, one of the most common cancers of dogs as well as humans. We have shown that canines treated with adriamycin or other antimetabolites have increased incidence of opportunistic microbial infections and that catechins stimulate the depressed immune response to such infections.

We have already shown that catechins, in a dose related manner, stimulate host immune responses, including soluble humoral factors such as cytokines and immune cells such as T cells, macrophages and dendritic cells. We are now analyzing the mechanism how catechins stimulate immune parameters when the immune system is compromised after administration of a chemotherapeutic antimetabolite such as adriamycin. Establishment of a nontoxic immunoenhancing adjuvant such as catechins for preventing the untoward immunosuppressive effects of antimetabolites in patients being treated for cancer has tremendous commercial biomedical potential. Thus, we will show that the use of green tea components, such as epigallocatechin gallate, when administered in conjunction with cancer chemotherapy can aid in the prevention of microbial infections and reduce the associated mobidity and mortality in both humans and animals.

### SUMMARY OF INVENTION

A combination cancer therapy based upon catechins, the major biologically active polyphenol in plant products, including green tea extract, and one or more chemotherapeutic agents. A common complication of cancer chemotherapy is neutropenia, and in spite of advances in its prophylactic management is a major cause of risk for the development of serious microbial infections leading to increased morbidity and mortality in both humans and animals. The use of cathechins such as those found in green tea (Camellia sinensis), including but not limited to epigallocatechin gallate (EGCG) as nontoxic adjuvant to aid in the prevention of opportunistic microbial infections in patients undergoing immunosuppressive chemotherapy is a novel application. Also contemplated are methods using catechins to ameliorate the immunosuppressive effects of cancer chemotherapy by administering the compound to a patient in need thereof.

In accordance with the present invention there is provided a combination cancer therapy comprising a catechin and one or more chemotherapeutic agents. In certain specific embodiments the one or more chemotherapeutic agents includes doxorubicin, vincristine sulfate, and/or cyclophosphamide. In certain specific embodiments the catechin is a camellia sinensis-derived catechin. In certain specific embodiments is a green tea-derived catechin. In certain specific embodiments the catechin is epigallocatechin gallate. In certain specific embodiments the catechin is Polyphenon 100. In certain specific embodiments the catechin comprises about 59.1% EGCg, about 1.6% GC, about 19.3% EGC, about 6.4% EG, and about 3.7% ECG.

The present invention also provides methods of treating a patient undergoing cancer chemotherapy to reduce the incidence of opportunistic microbial infections comprising the step of administering one or more catechins to a patient in need thereof. The methods can further include the step of administering one or more chemotherapeutic agents. In certain embodiments the chemotherapeutic agents can include doxorubicin, vincristine sulfate and/or cyclophosphamide. In certain specific embodiments the catechin is a camellia sinensis-derived catechin. In certain specific embodiments is a green tea-derived catechin. In certain specific embodiments the catechin is epigallocatechin gallate. In certain specific embodiments the catechin is Polyphenon 100. In certain specific embodiments the catechin comprises about 59.1% EGCg, about 1.6% GC, about 19.3% EGC, about 6.4% EG, and about 3.7% ECG.

The present invention also provides methods of reducing neutropenia in a patient undergoing cancer chemotherapy comprising the step of administering one or more catechins to a patient in need thereof. The methods can further include the step of administering one or more chemotherapeutic agents. In certain embodiments the chemotherapeutic agents can include doxorubicin, vincristine sulfate and/or cyclophosphamide. In certain specific embodiments the catechin is a camellia sinensis-derived catechin. In certain specific embodiments is a green tea-derived catechin. In certain specific embodiments the catechin is epigallocatechin gallate. In certain specific embodiments the catechin is Polyphenon 100. In certain specific embodiments the catechin comprises about 59.1% EGCg, about 1.6% GC, about 19.3% EGC, about 6.4% EG, and about 3.7% ECG.

### BRIEF DESCRIPTION OF THE DRAWINGS

For a fuller understanding of the invention, reference should be made to the following detailed description, taken in connection with the accompanying drawings, in which:

FIG. 1 is a diagram depicting the timeline for treatment including the chemotherapeutic agents administered and the times of administration for each of the administered agents. The figure highlights the aims of the experimental design including a daily measurement of rectal temperatures, the periods of remission and relapse and cytokine profiles taken during the course of the study.

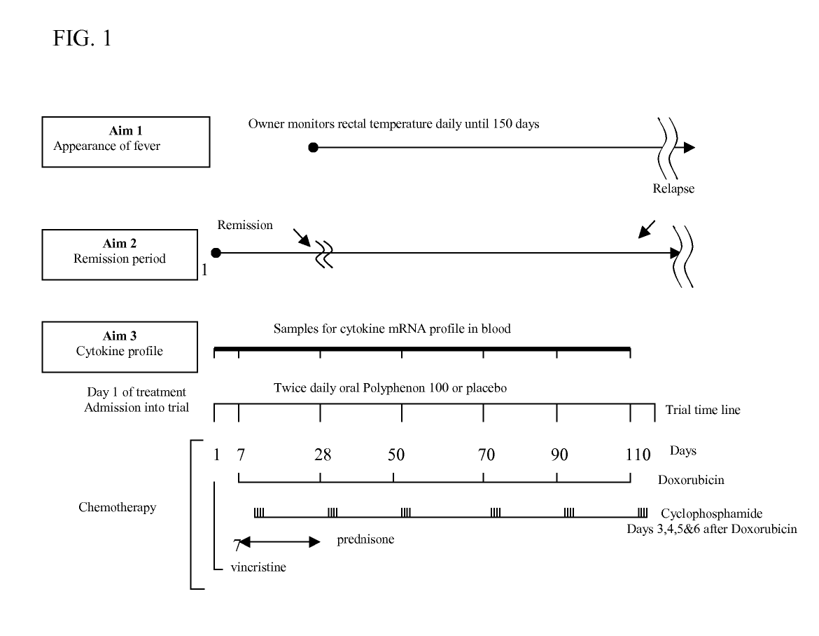

### DETAILED DESCRIPTION OF THE PREFERRED EMBODIMENT

Our product is a novel adjuvant for chemotherapy based upon catechins, the major biologically active polyphenol in plant products, including green tea extract. We have shown that catechins augment the biologic activity and this aspect will prove useful in cancer treatment based upon chemotherapy with toxic antimetabolites. The most common unwanted adverse reaction to antimetabolites is increased susceptibility to opportunistic microbial infection because of the known immunosuppressive properties of chemotherapeutic agents which markedly decrease the immune function. We have shown that catechins stimulate host immune responses to microbes. A combination of catechins with an immunosuppressive antimetabolite spares the immune system of the cancer host.

We will show the effectiveness of oral administration of a major component of green tea (Camellia sinerensis) in preventing opportunistic infections as a consequence of intrinsic chemotherapeutic immune suppression using pet dogs with naturally occurring neoplasias as a model. This is a novel approach for the evaluation of a complementary alternative medicine (CAM) in the context of immunesuppression and cancer prevention. Pet dogs diagnosed with cancer will be randomly assigned to one of two groups before commencing cancer treatment: 1) A control group receiving chemotherapy plus placebo and 2) a treatment group receiving the same chemotherapy plus oral administration of the green tea-derived catechin.

A common complication of cancer chemotherapy is neutropenia, and in spite of advances in its prophylactic management is a major cause of risk for the development of serious microbial infections leading to increased morbidity and mortality in both humans and animals. The use of cathechins such as those found in green tea (Camellia sinensis), including but not limited to epigallocatechin gallate (EGCG) as nontoxic adjuvant to aid in the prevention of opportunistic microbial infections in patients undergoing immunosuppressive chemotherapy is a novel application.

The invention will be further described by way of the following non-limiting examples.

### Example 1

### Development and Characterization of the Catechin Adjuvant for use in Cancer Chemotherapy

1. Trial Design

We propose to use a randomized double-blinded trial using dogs diagnosed with cancer and scheduled to receive treatment with chemotherapeutic agents known to induce neutropenia. By “chemotherapeutic agent” it is meant a drug used to treat cancer. Both the owner and the clinician examining and treating the dogs are masked with respect to treatment assignment. The control group receives chemotherapy treatment plus placebo in oral capsules and the treatment group receives chemotherapy plus catechin in oral capsules.

2. Sample Size and Data Analysis

Assuming a 10% placebo effect based on similar studies with natural products (Nagle et al., 2001; Sheehan & Atherton, 1992) 62 individuals per group are needed to detect a catechin response rate of 30% with 80% power using a significance level of 5% (two-sided) (Shuster, 1990; Nagle et. al, 2001). Assuming a 20% drop rate, 83 pet-dogs will be enrolled in each group. Based on three-year historical data of canine lymphomas at FVS the accrual period should be approximately six to eight months.

Allocation of the patients entering the trial will be done using a simple randomization method (pre-prepared randomization code) into placebo control and treatment groups (Shuster, 1990). The PI, Dr. van Olphen will act as the pharmacist and will be in charge of preparing and distributing the catechin and placebo to the clinician, who will ultimately provide them to the pet's owners. The catechin in the form of Polyphenon 100 and the placebo tablets will be dispense in identical plastic amber vial and labeled with a codified number known only to the pharmacist. All other participants will be masked.

Data are collected on standard data forms and entered into a computer program for analysis. Time to event analysis will be employed to evaluate the time to onset of fever for specific aim 1 and time to relapse in aim 2. All tests will be performed at the 5% significance level. Other statistical evaluation will be performed by the standard Student's t test and ANOVA statistical procedures.

3. Measurable Parameters and Methods

Aim 1: Time to appearance of fever: Period comprised from the day when chemotherapy is initiated to the day when fever is recorded for the first time. Fever is defined as temperature above 103° F. (39.4° C.) for at least two consecutive measurements taken 6 hours apart. The threshold value set for fever is equivalent to a minimum differential of 1.88° F. (1.0° C.) with respect to the values of normal diurnal temperature previously reported for beagle dogs (38.2±0.2° C. or 100.76±0.36° F.) (Miyazaki et al., 2002; Besch et al., 1984).

Monitoring of the rectal body temperature for the detection of fever will be done daily at the pet's home by the owner using a digital thermometer provided by the researchers. Thermometers will be tested before and during the trial to asses proper functioning. Owners will be asked to measure the rectal temperature in degrees Fahrenheit because it is the unit that they use in a routine basis. The researchers will also provide training for proper measurement of rectal temperature and a standard form for recording the patient's name, date, time, rectal temperature and comments on a daily basis (daily log). Monitoring of patient's body temperature will begin at the initiation of the oral administration of catechin or placebo and will continue until fever is recorded or for 30 days after the chemotherapy treatment. The treatment protocol includes the use of prednisone which most likely will prevent the appearance of fever during the first 4-5 weeks of chemotherapy and consequently we do not expect to observe a difference between groups. This period of temperature recordings before withdrawal from corticoids treatment will be use to correct potential technical difficulties encounter by the owner during measurement of the dog's rectal temperature. However, compliance is not a criterion for exclusion because is likely to affects both arms of the experiment (Shuster, 1990).

The following aims are secondary in terms of the statistical analysis.

Aim 2: Determine if administration of catechin extends the period comprised between treatment initiation and cancer relapse after the first remission. This period will be measured in days from the initiation of chemotherapy to the first relapse after the first cancer remission. Individuals that do respond to treatment (lack of clinical remission, partial remissions are included) will be excluded from the trial. The remission and relapse evaluation is performed by the clinician, who is masked for the catechin/placebo treatments. Remission is defined as the disappearance of recognizable superficial lymphadenopathy. Relapse is the reappearance of superficial lymphadenopathy. We recognize that cancer stage would have an impact in the treatment outcome, however by using a random patient allocation and excluding non-responders to treatment should help minimize this effect.

Aim 3: Determine the difference in specific cytokine profile at the transcription level (mRNA RT-PCR) between pet dogs in the control group versus those in the treatment group measured in PBMCs (buffy coat).

RT-PCR analysis: The following cytokines will be measured TNF-α, IL-2, IL-3, IFN-γ, IL-18, IL-4, IL-6 and IL-10. The rationale for selecting these cytokines for analysis is based on their important role in the regulation of the immune system and associated with immune mediated or inflammatory disorders (Mosmann & Sad, 1996). Cell-mediated immunity and macrophage (Cavaillon, 1994) activation are regulated by expression of the cytokines IFN-γ, IL-2 and IL-12 (Morel & Oriss, 1998). IL-4, IL-6 and IL-13 excerpt inhibitory effect on cell-mediated immunity in (Van der Meide & Schellekens, 1996) and together with IL-10, are able to amplify humoral response. IL-18, also known as the IFN-γ-inducing factor, acts as a co-stimulatory factor on Th1 cells, but not on Th2 cells, and shares with IL-12 the role of activating NK cells and biasing T cells toward Th1 cell function (Naume & Espevik, 1994).

Semi-quantitative analyses of cytokines will be performed using primers and methods essentially as described by Chamiso et. al (Chamizo et al., 2001) with the exception of IL-3 for which the primers used have been described by Shin et. al (Shin et al., 2001). Briefly, blood sample will be collected in vacuum tubes containing heparin (10 ml or more) and processes to separate peripheral blood mononuclear cells (PBMC). PBMC (4×10⁶ cells/2 ml) from the placebo and Catechin groups will be harvested on the days that patients receive doxorubicin prior to the administration for RNA extraction, and total cellular RNA will be prepared using TRIZOL reagent (GIBCO BRL) according to the manufacturer's instructions (Chomczynski, 1993). The prepared total RNA pellet will be dissolved in 20 μl water treated with diethylpyrocarbonate (DEPC) (2×10⁴ equivalent cells of RNA). Canine cytokine and GAPDH mRNA will be amplified from total RNA using reverse transcription and polymerase chain reaction (RT-PCR) (Shin et. al, 2001; Chamizo et. al, 2001; Klein et al., 2004; Lee et al., 2001). Reverse transcription will be performed at 60° C. for 60 min. Subsequent DNA amplification will be performed using an initial step of 96° C. for 1 min and a two-step amplification comprising two cycles of 94° C. for 1 min and 60° C. for 4 min, 35 cycles of 94° C. for 1 min and 60° C. for 2 min 30 s, then ending with one cycle of 72° C. for 8 min. Standard 2% agarose gel electrophoresis will be performed using 10 μl amplified samples and the intensities of ethidium bromide-stained bands analyzed using a Kodak Science ID image analysis system. Values with two fold increase or decrease, based on target/control ratios will be consider different.

4. Criteria to Enroll Patients in the Trial:

Dogs of any breed, sex, neutered or not, older than 12 month and diagnosed with lymphosarcoma scheduled to receive myelosuppressive chemotherapy (Northrup et al., 2002; Boyce & Kitchell, 2000) (see protocols below) will be eligible to enter in the trial.

Enrollment will be strictly voluntary, but the owner will be asked to sign an informed consent form.

5. Criteria for Exclusion from the Trial:

Patients that do not respond to cancer treatment (lack of remission) in either group will be excluded from the trial

Dogs weighting more than 60 kg will be excluded

Patients with known heart problems due the potential cardiac effects of chemotherapeutic drug (Doxorubicin)

Patients with known chronic systemic diseases (i.e. diabetes, hepatic dysfunction)

Dogs that have their treatment interrupted for reasons not directly associated with chemotherapy (i.e. financial, death due to an accident).

The owner will be asked to do not give any supplemental medication without previous consultation with the veterinary oncologist at FVS. However, if during the course of the trial a patient receives non-prescribed or doctor's recommended medication it will be removed from the trial.

6. Results, Pitfalls and Shortcomings

The most important measurable outcome of this trial is fever, as a marker of opportunistic infections, and the researchers will rely on pet owners to maintain daily recordings. We recognize this as weakness, and although overt infections would probably be a better parameter, the number of patients with identifiable infections is considerably lower that the incidence of fever.

Not all patients with lymphoma will be diagnosed and will start treatment at the same time and therefore are not expected to react to chemotherapy in the same way. In an attempt to reduce this effect without recurring to further stratification and having to either considerably increasethe sample size or reduce the power of the statistical analysis we have decided to exclude from the trial patients that do not respond in a clinically tangible manner to the instituted cancer treatment. In addition, our hypothesis is that catechins possess antimicrobial activity and that because of this fewer animals suffer opportunistic infections and have their treatments interrupted, aiding in consequence to a longer period before relapse. Although, as far as we know, there are no reports of positive effects of catechins against non-Hodgkin's lymphomas, EGCg has been reported to revert doxorubicin resistance tumors in an animal model (Zhang et al., 2004). The proposed mechanism is an increased concentration of doxorubicin in the tumors than in normal tissue, and therefore resistant tumors become susceptible to treatment. We are not proposing to evaluate this phenomenon in this proposal, but we expect that our results may suggest whether catechins have a potential to fight against lymphomas through this mechanism as well.

With the exception of the measurement of differentially cytokine expression between the two groups, we are not explicitly proposing to examine the molecular mechanisms by which catechins exert the proposed effect, however extensive clinical data will be collected to help focus future research directions.

7. Patient Care and Additional Considerations

The following considerations are provided to illustrate the type of procedures that pet dogs may be subjected to during the course of chemotherapy.

All study animals are examined weekly by a DVM at the Veterinary Clinic to determine the anti-tumor efficacy of the chemotherapy they are given. The animals have blood analysis performed on a weekly schedule or more frequently if warranted and the general health of the animals evaluated by a staff veterinarian. If the animal is losing weight, showing evidence of nausea or vomiting, etc., this will also be noted weekly by the veterinarian. This will also be done in consultation with the pet owner. Evaluation of efficacy of the chemotherapeutic treatment will be done weekly in the usual manner by examination by the veterinarian at the Clinic. Possible side effects of chemotherapy will be similarly evaluated weekly. During the weekly examinations, evidence of infection will be determined by standard means, i.e., loss of weight, listlessness of the animals, etc. Monitoring of the rectal temperature for the detection of fever will be done daily at the pet's home by the owner using a digital thermometer provided by the researchers. If an infection is suspected, blood samples are obtained for bacterial culture. Routine CBC is performed weekly for all animals and the animals carefully evaluated by standard diagnostic microbiologic laboratory criteria for evidence of infection. Evaluation for microbial infection, including that caused by opportunistic bacteria or fungi, is performed using accepted standard microbiologic techniques in the clinical laboratory. Antibiotic susceptibility tests of isolated organisms, once identified, are performed by routine microbiologic procedures.

Animal-Patient Population:

The animal patient population consists of canines referred to the Florida Veterinary Specialists and Cancer Treatment Center for putative multicentric lymphosarcoma. Over 300 dogs are treated per year at the Clinic for lymphosarcoma. Approximately 8-10 new cases are seen per week and about 6 of these dogs are put on chemotherapy immediately with the informed consent of the owner. Pretreatment and evaluation consist of physical examination, complete differential blood counts, urine analysis, serum biochemical profile, bone marrow aspiration cytology, thoracic radiography, abdominal ultrasonography, and electrocardiography. Diagnosis is established by lymph node aspirational cytology and/or lymph node biopsy specimens of affected nodes. The lymphomas are staged according to World Health Organization staging criteria for multicentric canine lymphosarcoma.

Catechin:

The product is Polyphenon 100, is marketed by Origin and contains 100 mg of catechins, consisting of 59.1% EGCg, 1.6% GC, 19.3% EGC, 6.4% EG, and 3.7% ECG and inactive ingredients (see below). Placebo tablets are obtained also from Mitsui Norin and contain only inactive ingredients (soybean oil, wheat germ oil, beeswax, gelatin emulsifier). The placebo and catechin tablets will be given to the control and treatment groups, respectively, orally twice a day beginning with the first doxorubicin treatment (7 days post treatment initiation) and will continue until the appearance of fever or until the end of the chemotherapeutic treatment (approximately day. 120). Compliance will be monitored during weekly visits to the clinic by reviewing the owner's records of treatment/placebo administration, and body temperature. In addition a plasma sample will be analyzed for EGCg content using HPLC at day 28 and 70.

Catechin dose:

The selection of the dosage is empirically set as follow:

The total number of tablets will be divided in a morning and evening dose (half and half). Each tablet contains approximately 60 mg of EGCg.

| Weight in | | maximum daily EGCg | Minimum daily EGCg |
|---|---|---|---|
| kg | Tablets a day | dose (mg/kg/day) | dose (mg/kg/day) |
| up to 15 | 2 | 60 | 8 |
| 15.1-30 | 4 | 16 | 8 |
| 30.1-60 | 6 | 12 | 6 |

Previous reports indicate in vivo therapeutic activity at a plasma concentration of 1 to 10 μM/L (Lambert & Yang, 2003). Swezey et. al (Swezey et al., 2003) studied the absorption, tissue distribution and elimination of 4-[³H]-epigallocatechin gallate in beagle dogs and estimated that approximately 20% of the oral dose appears in plasma after one hour and a half life of approximately 8 h. Considering an underestimation of this value to be 50 μg/ml and the molecular weight of EGCg of 458.4 g/L a daily dose of 250 mg/kg/day would result in a plasma concentration of approximately 100 μM/L. The doses proposed herein would result in an extrapolated value of more than 11 M/L in plasma.

EGCg plasma concentrations will be measured in plasma during the experiments to asses 1) owner's compliance and 2) to determine if the dose achieves the estimated concentrations in plasma. The measurement will be done in collaboration with Dr. Swezey from SRI International (a letter indicating the willingness to collaborate in this part of the project is included in section I).

Standard Chemotherapeutic Treatment:

Vincristin sulfate (0.7 mg/m2 of body surface BSA), administered i.v. on day one.

Prednisone given on days 7 through 28 (30, 20 and 10 mg/m2 BSA) PO, tapered weekly.

Doxorubicin hydrochloride (30 mg/m2 BSA, 20 min i.v. infusion) on day 7 and every 3 weeks for 6 treatments (total dose=180 mg/m2 BSA).

Cyclophosphamide (50 mg/m2 BSA, PO) on days 3, 4, 5, and 6, by owner after each doxorubicin treatment. (Boyce & Kitchell, 2000).

Treatment success is determined by prevention of morbidity, mortality, or both. Remission rates are approximately 75-80% (Mutsaers et al., 2002; Garrett et al., 2002; Boyce & Kitchell, 2000) with most patients having remission the first month of chemotherapy. Median survival first time after remission is 6-9 months. Common clinical problems during remission, are treated on an outpatient basis and occur in approximately 60% of patients (based on retrospective analysis of the FVS computer data base), including infections and anorexia, vomiting and diarrhea (similar to human patients treated with similar chemotherapy). Approximately 15% of patients require hospitalization during chemotherapy for treatment of septic shock. Treatment includes aggressive fluid and colloid therapy, broad spectrum antibiotics and supportive care. Tumor relapse is characterized by the patient showing again clinical disease, i.e., peripheral lymphadenopathy. A “rescue” protocol is often performed in attempts to re-induce remission and, generally, a second remission lasts half as long as the first remission. Progressive disease is manifested by lethargy, anorexia, weight loss and malaise.

Remissions occur in about 80% of the chemotherapy treated dogs and the usual length of time of remission is 6-9 months. Approximately 60% of the animals, once they develop progression of the disease again, are re-treated in an attempt to re-induce remission. Informed written consent of the owner is always obtained. Variables include number of neutropenic episodes, number of times of symptomatic therapy for infections when antibiotics are prescribed, comparison of median survival times, number of hospitalization days required for treatment of infectious diseases, assessment of life quality and comparison of median remission times and reason of death (i.e., due to progressive disease, due to complications of therapy, including infections, and decision for euthanasia of the pet due to complication of therapy).

Evaluation for therapy-toxicity

Clinical history, physical examination, complete differential blood count, urine analysis and serum biochemistry profile tests are performed prior to each scheduled doxorubicin treatment. An electrocardiogram or echocardiogram will be evaluated prior to each treatment. Doxorubicin therapy may be discontinued, if changes are consistent with cardiomyopathy, in which case the patient will be removed from the study. Standard anti-arrhythmic and other cardiac drugs may be administered as indicated. Dog diets are the same for the control and treated groups and previous studies have shown that changes in appetite of the chemotherapy treated dogs is not related to outcome. However, from this study, it will be possible to determine whether addition of the nutraceutical to the animal's diet will decrease the effect of the chemotherapy on the animal's well being and therefore improve their general physiological response as well as increase resistance to infections.

Opportunistic infection evaluation of therapy-side effects for incidence of infection is determined, and permission form completed and obtained.

Laboratory studies include the following:

Blood analysis.

Preparation of PBMCs, PMNs, lymphocytes and monocytes.

Cytokine mRNA expression in PMNs.

Infection Evaluation

All patients following chemotherapy becoming symptomatic for microbial infection are evaluated by determinators as follows:

a. Fever (primary measurable clinical outcome)

b. Purulent drainage

c. Leukocytosis or leukopenia

d. Hypoalbuminemia

e. Hypoglycemia

f. Positive blood and/urine cultures

Antimicrobial Therapy

As soon as patients are diagnosed as having an infection, they are placed on antibiotic therapy with any combination of fluoroquinolone, penicillin, cephalosporin, aminoglycoside, metronitrazole, and macroglide antibiotics.

The most common bacteria cultured from symptomatic animals result from GI tract translocation and the most common bacteria include:

a) anaerobic bacteria, colon bacteroides spp (23% of anaerobes isolated)

b. Peptostreptococcus spp (8% of anaerobes)

c. Fusobacterium spp (14% of anaerobes)

d. Pseudomonas spp (11% of anaerobes)

e. Some Gram negative bacteria (E. coli, Klebsiella spp and Proteus, spp)

f. Gram positive aerobic infections are usually uncommon.

g. Fungal, viral and parasitic infections are addressed prior to onset of chemotherapy and do not cause a problem during chemotherapy treatment in dogs.

### REFERENCE LIST

Besch, E. L., Kadono, H., and Brigmon, R. L. (1984). Body temperature changes in dogs exposed to varying effective temperatures. Lab Anim Sci. 34, 177-180.
Boyce, K. L. and Kitchell, B. E. (2000). Treatment of canine lymphoma with COPLA/LVP. J. Am.Anim Hosp.Assoc. 36, 395-403.
Cavaillon, J. M. (1994). Cytokines and macrophages. Biomed.Pharmacother. 48, 445-453.
Chamizo, C., Rubio, J. M., Moreno, J., and Alvar, J. (2001). Semi-quantitative analysis of multiple cytokines in canine peripheral blood mononuclear cells by [correction of zby] a single tube RT-PCR. Vet.Immunol.Immunopathol. 83, 191-202.
Chomczynski, P. (1993). A reagent for the single-step simultaneous isolation of RNA, DNA and proteins from cell and tissue samples. Biotechniques 15, 532-537.
Garrett, L. D., Thamm, D. H., Chun, R., Dudley, R., and Vail, D. M. (2002). Evaluation of a 6-month chemotherapy protocol with no maintenance therapy for dogs with lymphoma. J. Vet.Intern.Med. 16, 704-709.
Klein, T. W., Newton, C., Larsen, K., Chou, J., Perkins, I., Lu, L., Nong, L., and Friedman, H. (2004). Cannabinoid receptors and T helper cells. J. Neuroimmunol. 147, 91-94.
Lambert, J. D. and Yang, C. S. (2003). Mechanisms of cancer prevention by tea constituents. J Nutr. 133, 3262S-3267S.
Lee, S. F., Newton, C., Widen, R., Friedman, H., and Klein, T. W. (2001). Differential expression of cannabinoid CB(2) receptor mRNA in mouse immune cell subpopulations and following B cell stimulation. Eur.J Pharmacol. 423, 235-241.
Miyazaki, H., Yoshida, M., Samura, K., Matsumoto, H., Ikemoto, F., and Tagawa, M. (2002). Ranges of diurnal variation and the pattern of body temperature, blood pressure and heart rate in laboratory beagle dogs. Exp.Anim 51, 95-98.
Morel, P. A. and Oriss, T. B. (1998). Crossregulation between Th1 and Th2 cells. CritRev.Immunol. 18, 275-303.
Mosmann, T. R. and Sad, S. (1996). The expanding universe of T-cell subsets: Th1, Th2 and more. Immunol.Today 17, 138-146.
Mutsaers, A. J., Glickman, N. W., DeNicola, D. B., Widmer, W. R., Bonney, P. L., Hahn, K. A., and Knapp, D. W. (2002). Evaluation of treatment with doxorubicin and piroxicam or doxorubicin alone for multicentric lymphoma in dogs. J Am Vet.Med.Assoc. 220, 1813-1817.
Nagle, T. M., Torres, S. M., Home, K. L., Grover, R., and Stevens, M. T. (2001). A randomized, double-blind, placebo-controlled trial to investigate the efficacy and safety of a Chinese herbal product (P07P) for the treatment of canine atopic dermatitis. Vet.Dermatol. 12, 265-274.
Naume, B. and Espevik, T. (1994). Immunoregulatory effects of cytokines on natural killer cells. Scand.JImmunol. 40, 128-134.
Northrup, N. C., Rassnick, K. M., Snyder, L. A., Stone, M. S., Kristal, O., Cotter, S. M., and Moore, A. S. (2002). Neutropenia associated with vincristine and L-asparaginase induction chemotherapy for canine lymphoma. J. Vet.Intern.Med. 16, 570-575.
Sheehan, M. P. and Atherton, D. J. (1992). A controlled trial of traditional Chinese medicinal plants in widespread non-exudative atopic eczema. Br.J. Dermatol. 126, 179-184.
Shin, I. S., Kim, H. R., Nam, M. J., and Youn, H. Y. (2001). Studies of cocktail therapy with multiple cytokines for neoplasia or infectious disease of the dog I. cDNA cloning of canine IL-3 and IL-6. J. Vet.Sci. 2, 115-120.
Shuster, J. J. (1990). “Handbook of sample size guidelines for clinical trials.” CRC Press, Boca Raton, Fla.
Swezey, R. R., Aldridge, D. E., LeValley, S. E., Crowell, J. A., Hara, Y., and Green, C. E. (2003). Absorption, tissue distribution and elimination of 4-[(3)h]-epigallocatechin gallate in beagle dogs. Int.J. Toxicol. 22, 187-193.
Van der Meide, P. H. and Schellekens, H. (1996). Cytokines and the immune response. Biotherapy 8, 243-249.
Zhang, Q., Wei, D., and Liu, J. (2004). In vivo reversal of doxorubicin resistance by (−)-epigallocatechin gallate in a solid human carcinoma xenograft. Cancer Lett. 208, 179-186.

The disclosure of all publications cited above are expressly incorporated herein by reference, each in its entirety, to the same extent as if each were incorporated by reference individually.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween. Now that the invention has been described,

What is claimed is:

1. A method of treating a patient undergoing cancer chemotherapy to reduce the incidence of opportunistic microbial infections comprising the step of administering one or more catechins to a patient in need thereof.

2. The method of claim 1 further comprising the step of administering one or more chemotherapeutic agents.

3. The method of claim 2 where the one or more chemotherapeutic agents is selected from the group consisting of doxorubicin, vincristine sulfate and cyclophosphamide.

4. The method of claim 1 wherein the catechin is a camellia sinensis-derived catechin.

5. The method of claim 1 wherein the catechin is a green tea-derived catechin.

6. The method of claim 1 wherein the catechin is epigallocatechin gallate.

7. The method of claim 1 wherein the catechin comprises about 59.1% EGCg, about 1.6% GC, about 19.3% EGC, about 6.4% EG, and about 3.7% ECG.

8. A method of reducing neutropenia in a patient undergoing cancer chemotherapy comprising the step of administering one or more catechins to a patient in need thereof.

9. The method of claim 8 further comprising the step of administering one or more chemotherapeutic agents.

10. The method of claim 9 where the one or more chemotherapeutic agents is selected from the group consisting of doxorubicin, vincristine sulfate and cyclophosphamide.

11. The method of claim 8 wherein the catechin is a camellia sinensis-derived catechin.

12. The method of claim 8 wherein the catechin is epigallocatechin gallate.

13. A combination cancer therapy comprising a catechin and one or more chemotherapeutic agents.

14. The combination cancer therapy of claim 13 wherein the one or more chemotherapeutic agents comprises doxorubicin.

15. The combination cancer therapy of claim 13 wherein the one or more chemotherapeutic agents is selected from the group consisting of vincristine sulfate, cyclophosphamide and doxorubicin.

16. The combination cancer therapy of claim 13 wherein the one or more chemotherapeutic agents is an antimetabolite.

17. The combination cancer therapy of claim 13 wherein the catechin is a camellia sinensis-derived catechin.

18. The combination cancer therapy of claim 13 wherein the catechin is a green tea-derived catechin.

19. The combination cancer therapy of claim 13 wherein the catechin is epigallocatechin gallate.

20. The combination cancer therapy of claim 13 wherein the catechin is Polyphenon 100.

21. The combination cancer therapy of claim 13 wherein the catechin comprises about 59.1% EGCg, about 1.6% GC, about 19.3% EGC, about 6.4% EG, and about 3.7% ECG.
